(19) United States
(12) Patent Application Publication
(10) Pub. No.: US 2016/0029886 A1
(43) Pub. Date: Feb. 4, 2016

(54) OPTICAL COHERENCE TOMOGRAPHY APPARATUS AND FUNDUS IMAGE PROCESSING PROGRAM

(71) Applicant: NIDEK CO., LTD., Gamagori (JP)

(72) Inventors: Ryosuke SHIBA, Gamagori (JP); Tetsuya KANOU, Toyota (JP); Norimasa SATAKE, Nukata (JP)

(21) Appl. No.: 14/814,145

(22) Filed: Jul. 30, 2015

(30) Foreign Application Priority Data
Jul. 31, 2014 (JP) 2014-157181

(51) Int. Cl. A61B 3/10; A61B 3/12

(52) U.S. Cl. CPC A61B 3/102; A61B 3/1025; A61B 3/12

(57) ABSTRACT

An optical coherence tomography apparatus includes: a scanning unit configured to scan a test substance with measurement light; an OCT optical system configured to detect an A scan signal caused by interference between the measurement light and reference light corresponding to the measurement light; and a processor configured to: acquire three-dimensional OCT data in which the A scan signals at respective scanning positions at the test substance are arranged in a two-dimensional manner; acquire a histogram of the A scan signals at the respective scanning positions; and generate an OCT front image by processing the acquired three-dimensional OCT data based on the acquired histogram.

### CROSS REFERENCE TO RELATED APPLICATION

This application is based upon and claims the benefit of priority of Japanese Patent Application No. 2014-157181 filed on Jul. 31, 2014, the contents of which are incorporated herein by reference in its entirety.

### BACKGROUND

The present disclosure relates to an optical coherence tomography apparatus and a fundus image processing program for obtaining a tomographic image of a test substance.

An optical coherence tomography (OCT) apparatus used to acquire a tomographic image of a test substance is known. Such an apparatus is used to obtain a tomographic image of a part of biological tissue such as the eye or skin. For example, in an ophthalmic medical field, a subject's eye is diagnosed based on a tomographic image of an ocular tissue (for example, the retina or the anterior chamber) obtained by the OCT apparatus.

In recent years, in addition to a B scan image, a technique for acquiring a front image (a so-called En face image) obtained when at least a part of the tissue of a biological tissue is viewed from a front direction has been proposed. For example, the front image is generated as an image by integrating three-dimensional OCT data regarding at least a partial region in a depth direction (refer to Specification of U.S. Pat. No. 7,301,644). In this case, luminance values of all A scan signals regarding a specific region which is selected in advance are integrated.

### SUMMARY

Methods in the related art may be insufficient in terms of image quality or a means of viewing a tissue. In addition, there is a possibility that a signal indicating abnormality such as a disease may be buried in other signals, and thus the disease may be hard to specify.

An object of the present disclosure is to provide an optical coherence tomography apparatus and a fundus image processing program, capable of solving at least one of the problems of the related art.

In order to solve the above-described problems, the present disclosure has the following configurations.

An optical coherence tomography apparatus comprising:

a scanning unit configured to scan a test substance with measurement light;

an OCT optical system configured to detect an A scan signal caused by interference between the measurement light and reference light corresponding to the measurement light; and

a processor configured to:

acquire three-dimensional OCT data in which the A scan signals at respective scanning positions at the test substance are arranged in a two-dimensional manner;

acquire a histogram of the A scan signals at the respective scanning positions; and

generate an OCT front image by processing the acquired three-dimensional OCT data based on the acquired histogram.

A non-transitory computer readable recording medium storing a fundus image processing program, when executed by a processor of an optical coherence tomography apparatus including a scanning unit configured to scan a test substance with measurement light and an OCT optical system configured to detect an A scan signal caused by interference between the measurement light and reference light corresponding to the measurement light, causing the optical coherence tomography apparatus to execute:

acquiring three-dimensional OCT data in which the A scan signals at respective scanning positions at the test substance are arranged in a two-dimensional manner;

acquiring a histogram of the A scan signals at the respective scanning positions; and

generating an OCT front image by processing the acquired three-dimensional OCT data based on the acquired histogram.

### BRIEF DESCRIPTION OF THE DRAWINGS

FIG. 1 is a block diagram illustrating a configuration of an optical coherence tomography apparatus according to the present embodiment;

FIG. 2 is a schematic diagram illustrating an OCT optical system;

FIGS. 3A and 3B schematically illustrate an acquired front image and tomographic image;

FIG. 4 is a diagram illustrating an example of a histogram acquired based on an A scan signal;

FIG. 5 is a diagram illustrating a setting screen for obtaining a front image, and a front image acquired through a setting process;

FIG. 6 is a flowchart illustrating an example in which a front image is obtained by using a histogram;

FIG. 7 is a diagram illustrating a method of setting a depth region for acquiring a histogram;

FIG. 8 is a diagram illustrating a depth region including a lesion portion;

FIG. 9 is a diagram illustrating a histogram obtained by using an A scan signal for the depth region including the lesion portion;

FIG. 10 is a diagram illustrating a front image of the fundus including the lesion portion;

FIG. 11 is a diagram illustrating an example of a method of setting a bin width;

FIG. 12 is a diagram illustrating an example of selecting a representative luminance value used to generate a front image;

FIG. 13 is a diagram illustrating an example of selecting a representative luminance value used to generate a front image;

### DESCRIPTION OF ILLUSTRATIVE EMBODIMENTS

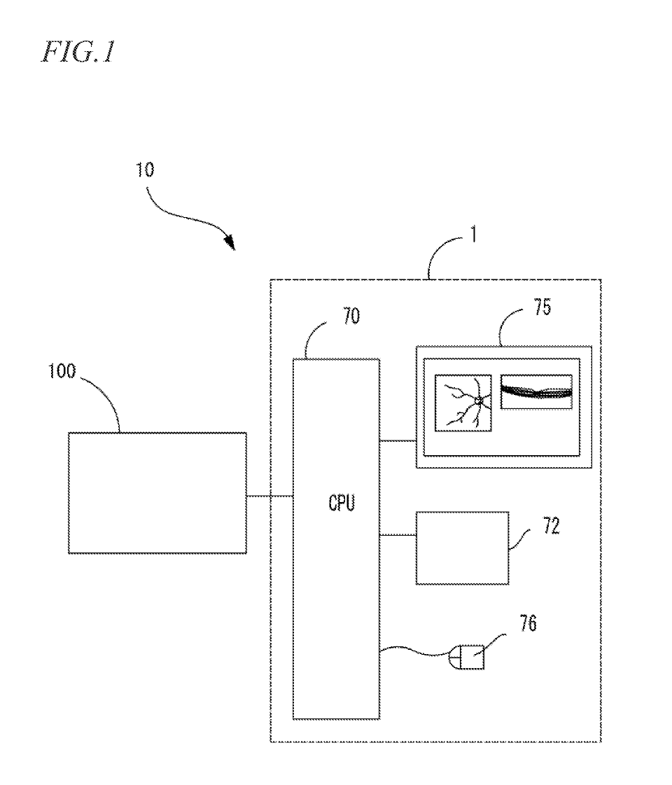

Hereinafter, a preferred embodiment will be described with reference to the drawings. FIG. 1 is a block diagram illustrating a configuration of an optical coherence tomography apparatus (hereinafter, also referred to as the present apparatus) 10 according to the present embodiment. As an example of the present apparatus 10, a fundus imaging apparatus which acquires a tomographic image of the fundus of a subject's eye will be described.

An OCT device 1 illustrated in FIG. 1 processes a detection signal acquired by an OCT optical system 100. The OCT device 1 includes a control unit 70. The OCT optical system 100 captures, for example, a tomographic image of the fundus Ef of a subject's eye E. The OCT optical system 100 is connected to, for example, the control unit 70.

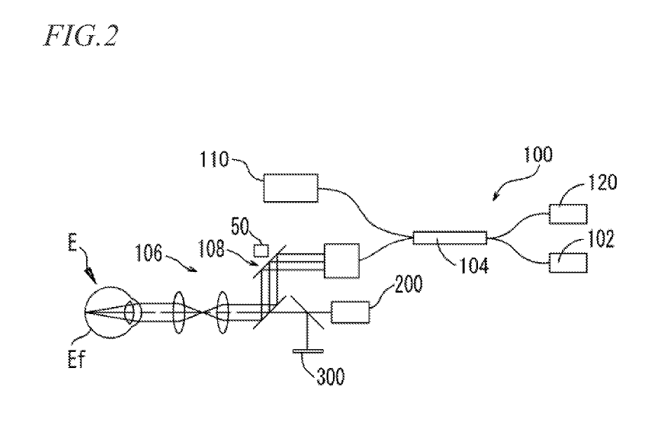

Next, the OCT optical system 100 will be described with reference to FIG. 2. The OCT optical system 100 irradiates the fundus with measurement light. The OCT optical system 100 detects an interference state between the measurement light reflected from the fundus and reference light by using a light receiving element (a detector 120). The OCT optical system 100 includes irradiation position changing units (for example, an optical scanner 108 and a fixation target projection unit 300) which change an irradiation position of measurement light on the fundus Ef in order to change an imaging position on the fundus Ef. The control unit 70 controls operations of the irradiation position changing units based on set imaging position information, and acquires a tomographic image based on a light reception signal from the detector 120.

<OCT Optical System>

The OCT optical system 100 has a so-called optical coherence tomography (OCT) configuration and captures a tomographic image of the eye E. The OCT optical system 100 splits light emitted from a measurement light source 102 into measurement light (sample light) and reference light by using a coupler (beam splitter) 104. The OCT optical system 100 guides the measurement light to the fundus Ef of the eye E by using a measurement optical system 106 and guides the reference light to a reference optical system 110. Then, interference light obtained by combining the measurement light reflected from the fundus Ef and the reference light is received by the detector (light receiving element) 120.

The detector 120 detects an interference signal between the measurement light and the reference light. In a case of a Fourier-domain OCT, a spectral intensity (spectral interference signal) of the interference light is detected by the detector 120, and a complex OCT signal is acquired through Fourier transform on the spectral intensity data. For example, an A scan signal (depth profile) is acquired by calculating an absolute value of the amplitude of the complex OCT signal. OCT data (tomographic image data) is acquired by arranging the A scan signals at respective scanning positions of measurement light emitted from the optical scanner 108. As mentioned above, the optical scanner 108 functions as a scanning unit.

Three-dimensional OCT data is acquired by performing scanning with the measurement light in a two-dimensional manner, and an OCT front image (En face image) is acquired by using the three-dimensional OCT data. In this case, the control unit 70 functions as a processor which generates an OCT front image by using three-dimensional OCT data.

As the OCT optical system 100, a spectral-domain OCT (SD-OCT) optical system may be used, and a swept-source OCT (SS-OCT) which detects a spectrum of interference light by using a wavelength variable light source changing the wavelength of emitted light may be used. Of course, a time-domain OCT may be used.

In a case of the SD-OCT, a low coherent light source (a wide area light source) is used as the light source 102, and the detector 120 is provided with a spectral optical system (spectrometer) which separates interference light into respective frequency components (respective wavelength components). The spectrometer is constituted of, for example, a diffraction grating and a line sensor.

In a case of the SS-OCT, a wavelength scanning type light source (wavelength variable light source) which changes emitted wavelengths temporally at a high speed is used as the light source 102, and, for example, a single light receiving element is provided as the detector 120. The light source 102 is constituted of, for example, a light source, a fiber ring resonator, and a wavelength selection filter. As the wavelength selection filter, for example, a combination of a diffraction grating and a polygon mirror, or one using a Fabry-Perot etalon may be used.

Light emitted from the light source 102 is split into measurement light beams and reference light beams by the coupler 104. The measurement light beams pass through an optical fiber and are emitted to air. The light beams are collected at the fundus Ef via the optical scanner 108 and other optical members of the measurement optical system 106. Light reflected from the fundus Ef is returned to the optical fiber along the same optical path.

The optical scanner 108 scans the fundus with measurement light in a two-dimensional manner (XY directions (crossing directions)). The optical scanner 108 is disposed at a position substantially conjugate to the pupil. The optical scanner 108 is constituted of, for example, two galvano mirrors, and reflection angles thereof are arbitrarily adjusted by a driving mechanism 50.

Consequently, reflection (traveling) directions of light beams emitted from the light source 102 are changed, and the fundus is scanned with the light beams at arbitrary position. Thus, an imaging position on the fundus Ef is changed. The optical scanner 108 may have a configuration of deflecting light. For example, not only a reflective mirror (a galvano mirror, a polygon mirror, or a resonant scanner) but also an acousto-optical modulator (AOM) which changes a traveling (deflection) direction of light is used.

The reference optical system 110 generates reference light which is combined with reflected light obtained by reflection of measurement light on the fundus Ef. The reference optical system 110 may be of a Michelson type, and may be of a Mach-Zenhder type. The reference optical system 110 is constituted of for example, a reflection optical system (for example, a reference mirror), and reflects light from the coupler 104 with the reflection optical system so that the light is returned to the coupler 104 and is thus guided to the detector 120. As another example, the reference optical system 110 is constituted of a transmission optical system (for example, an optical fiber), and transmits light from the coupler 104 through the transmission optical system without returning the light so that the light is guided to the detector 120.

The reference optical system 110 has a configuration of changing an optical path length difference between measurement light and reference light by moving an optical member on a reference optical path. For example, the reference mirror is moved in an optical axis direction. The configuration of changing an optical path length difference may be disposed on a measurement optical path of the measurement optical system 106.

<Front Observation Optical System>

The front observation optical system 200 is provided to obtain a front image of the fundus Ef. The front observation optical system 200 includes, for example, an optical scanner which scans the fundus with measurement light (infrared light) emitted from a light source in a two-dimensional manner, and a second light receiving element which receives light reflected from the fundus via a confocal aperture which is disposed at a position substantially conjugate to the fundus, and has a scanning laser ophthalmoscope (SLO) configuration.

In addition, a configuration of the front observation optical system 200 may be a so-called fundus camera type configuration. Further, the OCT optical system 100 may also be used as the observation optical system 200.

<Fixation Target Projection Unit>

The fixation target projection unit 300 includes an optical system for guiding a visual line direction of the eye E. The fixation target projection unit 300 has a fixation target presented to the eye E, and can guide the eye E in a plurality of directions.

<Control Unit>

The control unit 70 includes a CPU (processor), a RAM, a ROM, and the like. The CPU of the control unit 70 controls the entire apparatus (the OCT device 1 and the OCT optical system 100) such as the respective members of each configuration. The RAM temporarily stores various information pieces. The ROM of the control unit 70 stores various programs, initial values, and the like for controlling an operation of the entire apparatus. The control unit 70 may be constituted of a plurality of control units (that is, a plurality of processors).

The control unit 70 is electrically connected to a nonvolatile memory (storage means) 72, an operation unit 76, a display unit (monitor) 75, and the like. The nonvolatile memory (memory) 72 is a non-transitory storage medium which can hold storage content even if power is not supplied. For example, a hard disk drive, a flash ROM, and a USB memory which is attachable to and detachable from the OCT device 1 and the OCT optical system 100 may be used as the nonvolatile memory 72. The memory 72 stores an imaging control program for controlling the OCT optical system 100 to capture a front image and a tomographic image. In addition, the memory 72 stores a signal processing program which enables an OCT signal obtained by the OCT device 1 to be processed. Further, the memory 72 stores various information pieces regarding imaging, such as information regarding imaging positions on a scanning line for a tomographic image (OCT data), a three-dimensional tomographic data (three-dimensional OCT data), a fundus front image, and a tomographic image. An examiner inputs various operation instructions to the operation unit 76.

The operation unit 76 outputs a signal corresponding to an input operation instruction to the control unit 70. The operation unit 76 may employ at least one of, for example, a mouse, a joystick, a keyboard, and a touch panel.

The monitor 75 may be a display mounted in the apparatus main body, and may be a display connected to the main body. A display of a personal computer (“PC”) may be used. A plurality of displays may be used. The monitor 75 may be a touch panel. If the monitor 75 is a touch panel, the monitor 75 functions as an operation unit. Various images including a tomographic image and a front image captured by the OCT optical system 100 are displayed on the monitor 75.

A description will be made of the summary of an operation of the apparatus having the above-described configuration. The control unit 70 processes spectral data detected by the detector 120, and forms a fundus tomographic image and front image through image processing. The tomographic image and the front image may be acquired together, alternately, or sequentially. In other words, the spectral data may be used to acquire at least one of the tomographic image and the front image.

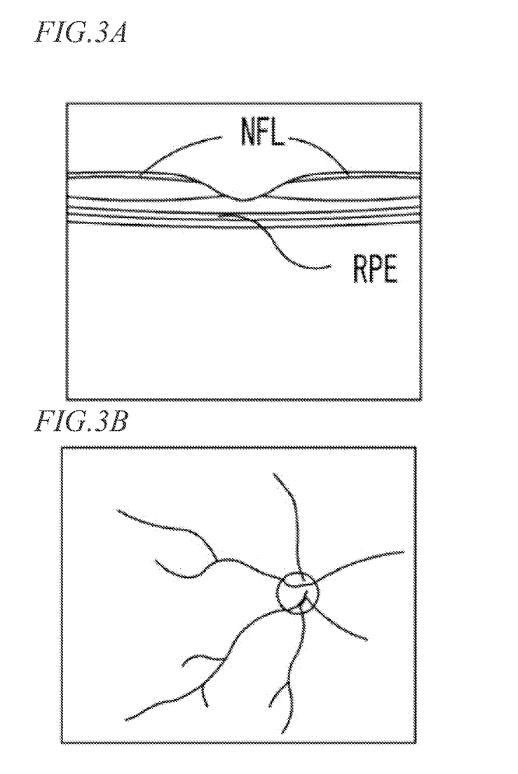

In a case of obtaining the tomographic image, the control unit 70 causes the driving mechanism 50 to scan the fundus Ef with measurement light in a crossing direction. The control unit 70 detects spectral data output from the detector 120 in relation to each scanning position (X and Y) on the fundus, transforms interference signals included in the detected spectral data into A scan signals, and arranges the A scan signals in the scanning direction so as to form the fundus tomographic image (refer to FIG. 3(a)). Here, the A scan signal is a signal indicating, for example, an interference intensity distribution of a test substance in the depth direction, and forms a column of luminance values in the depth direction.

An interference signal is extracted from the spectral data through a noise removal process, and the spectral data is transformed into an A scan signal by analyzing an amplitude level for each frequency (wave number) of the interference signal. A representative of the frequency analysis is Fourier transform. As a scanning pattern of measurement light, for example, a line pattern, a cross line pattern, a raster pattern, a circular pattern, or a radial pattern may be employed. Scanning is performed with the measurement light in a two-dimensional manner, and thus three-dimensional OCT data is acquired.

In a case of obtaining the tomographic image, the control unit 70 causes the driving mechanism 50 to scan the fundus Ef with measurement light in XY directions. Consequently, three-dimensional OCT data in which A scan signals at respective scanning positions are arranged in a two-dimensional manner is acquired. The control unit 70 creates a histogram of the A scan signals at the respective positions (X,Y), and obtains the front image of the test substance in the XY directions based on the histogram (refer to FIG. 3(b)). For example, the control unit 70 may acquire luminance values at the scanning positions (X,Y) based on a distribution of the histogram or a change in the histogram. The histogram obtained in the present embodiment is a histogram regarding a frequency (frequency of occurrence) of a luminance value forming an A scan signal.

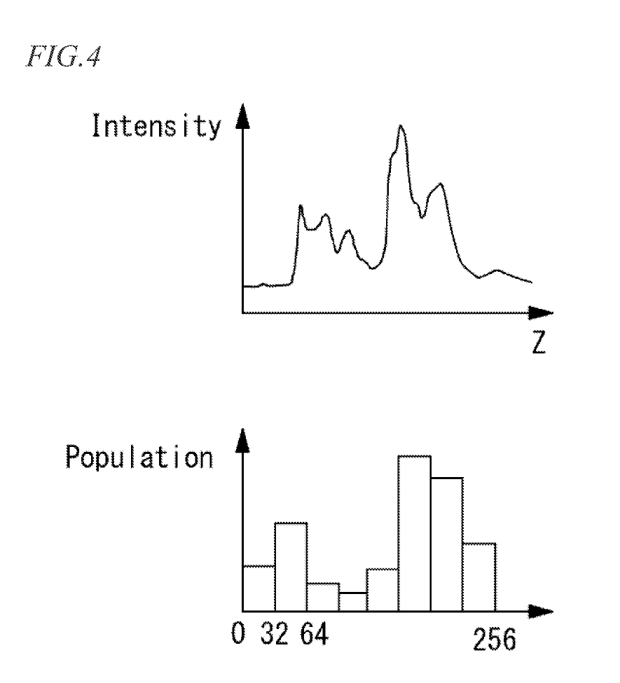

FIG. 4 is a diagram illustrating an example of a histogram acquired based on an A scan signal. The upper diagram is a graph illustrating an example of a signal intensity distribution of an A scan signal in the Z direction (transverse axis: Z direction (depth direction), and longitudinal axis: signal intensity). The lower diagram is a graph obtained when the signal intensity distribution of the upper diagram is represented by a histogram (transverse axis: luminance range, and longitudinal axis: frequency).

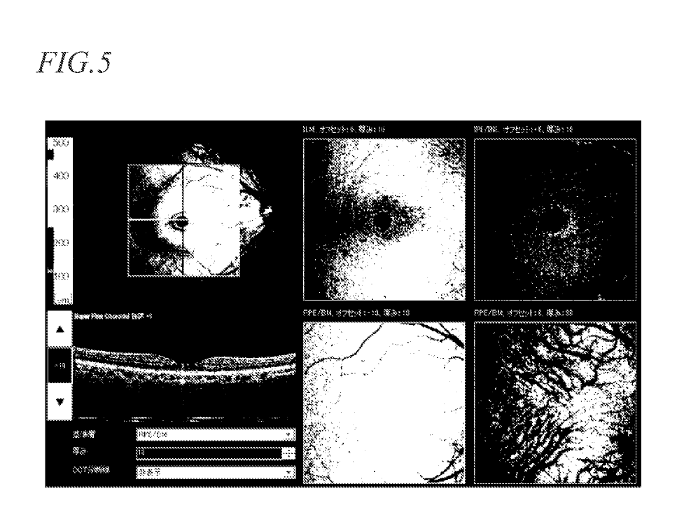

Hereinafter, a detailed description will be made of a method of acquiring a front image based on a histogram of the A scan signal. FIG. 5 is a diagram illustrating a setting screen for obtaining a front image, and a front image acquired through a setting process. On the setting screen, the examiner can arbitrarily set a reference layer for obtaining a front image, a thickness of the depth region which is set for obtaining a front image, a distance between the depth region and the reference layer, which is set for obtaining a front image, the presence or absence of an OCT dividing line for division segmentation, and the like. In other words, in the present embodiment, it is possible to acquire a front image based on a specific layer.

In FIG. 5, a plurality of (for example, four) front images are displayed, and the examiner can perform setting on the setting screen in relation to each front image, and can change a depth region regarding each front image. In addition, front images of a plurality of depth regions which are set in advance may be displayed separately, and the depth region set in advance may be changed on the setting screen.

As a depth region for acquiring a front image, all depth regions (all A scan signals) for forming three-dimensional OCT data may be set, and some depth regions (some A scan signals) for forming three-dimensional OCT data may be set.

On the setting screen illustrated in FIG. 5, a layer boundary between the retinal pigment epithelium layer (RPE layer) and the Bruch membrane (BM) is set as the reference layer, 10 pixels is set as a thickness of the depth region, and −10 pixels is set as the distance to the reference layer. Of course, these set values are only examples.

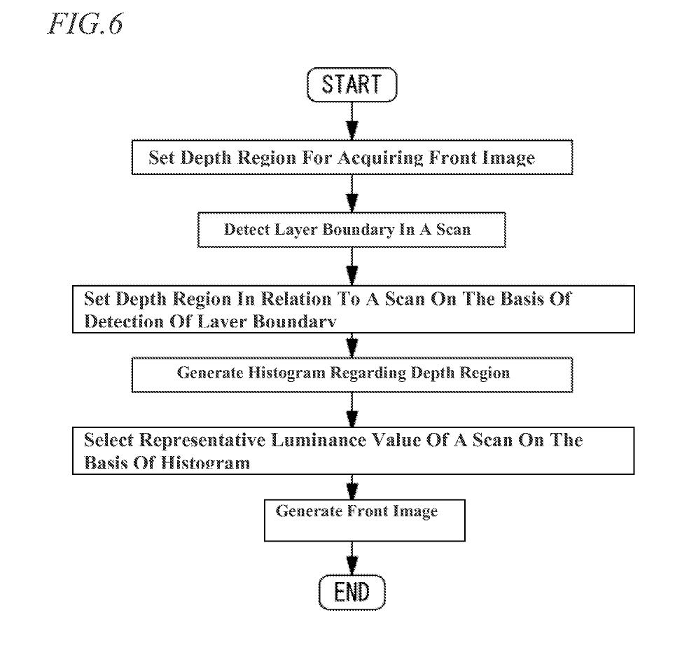

FIG. 6 is a flowchart illustrating an example of a process of obtaining a front image by using a histogram. The control unit 70 sets a depth region for acquiring a histogram of the A scan signal based on the set condition which is set as described above. FIG. 6 illustrates an example of a case of obtaining a histogram based on the set condition illustrated in FIG. 5.

Hereinafter, a description will be made of a case where a front image of a specific depth region is generated by obtaining a histogram of the A scan signal in the specific depth region. For example, the control unit 70 performs a division process on the A scan signal so as to detect a layer boundary of the fundus. In this case, the control unit 70 may detect a layer boundary corresponding to a specific layer (for example, the nerve fiber layer (NFL), the ganglion cell layer (GCL), or the retinal pigment epithelium (RPE)) through the division process. In a case of detecting a layer boundary corresponding to a specific layer, a detection method is set based on a position of the specific layer in terms of anatomy, an order of layers, a luminance level of the A scan signal, and the like. For example, edge detection is used for the division.

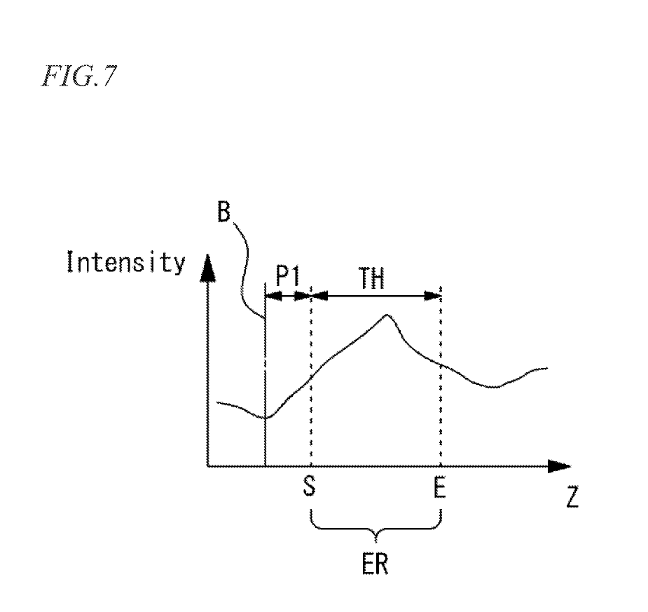

After the layer boundary corresponding to the specific layer is detected, as illustrated in FIG. 7, the control unit 70 sets a depth region ER which has a start point S far from the detected layer boundary B by P1 and an end point E at a position which is added by a thickness TH from the start point S. It is possible to acquire a favorable front image even if there is the occurrence of a variation in the layer boundary detection by using the A scan signal regarding the depth region far from the layer boundary. Of course, as the depth region ER, a depth region from a first layer boundary to a second layer boundary may be set.

The control unit 70 obtains a histogram regarding the set depth region ER through A scan. For example, the control unit 70 divides a luminance range into a predetermined number of sections (hereinafter, bins) in relation to a luminance distribution of the A scan signal in the depth region ER, and measures a frequency (frequency of occurrence) of luminance values corresponding to each bin. The control unit 70 generates a histogram regarding the luminance values based on a measurement result (refer to FIG. 4).

As a bin width is increased, an influence of noise can be reduced, but if a bin width is large, fine information is erased, and thus a grayscale range of a generated image is reduced. The bin width is set to an appropriate size based on such circumstances, test results, and the like. In FIG. 4, the bin width is set to 32 but is not limited thereto.

Next, the control unit 70 obtains a representative luminance value of the A scan signal based on the generated histogram. For example, the control unit 70 sets the median of a bin with the highest frequency of occurrence as a representative luminance value of the A scan based on the generated histogram, and uses the representative luminance value as a luminance value of a front image. The control unit 70 obtains a representative luminance value for each scanning position (X,Y) according to the above-described method. The control unit 70 uses each obtained representative luminance value as a luminance value of each pixel of a front image, so as to generate a front image as illustrated in FIG. 3(b). More specifically, the control unit 70 changes each luminance value at X and Y positions on the front image based on the representative luminance value obtained for each scanning position (X,Y).

As described above, a luminance value of each pixel forming an OCT front image is changed based on the change of a luminance value ranked high in the frequency on the histogram, and thus a front image is formed based on the dominant luminance information in the A scan signal. Thus, it is possible to acquire a favorable front image with a reduced influence of noise.

A luminance value column acquired using the OCT includes a high level of noise. This noise is shown as a random luminance value in the luminance value column, and thus there is a low possibility that the frequency of occurrence of a specific luminance value may increase due to the noise.

The histogram acquired in the above-described way changes depending on an object which forms a fundus region corresponding to the depth region ER which is set to acquire a front image.

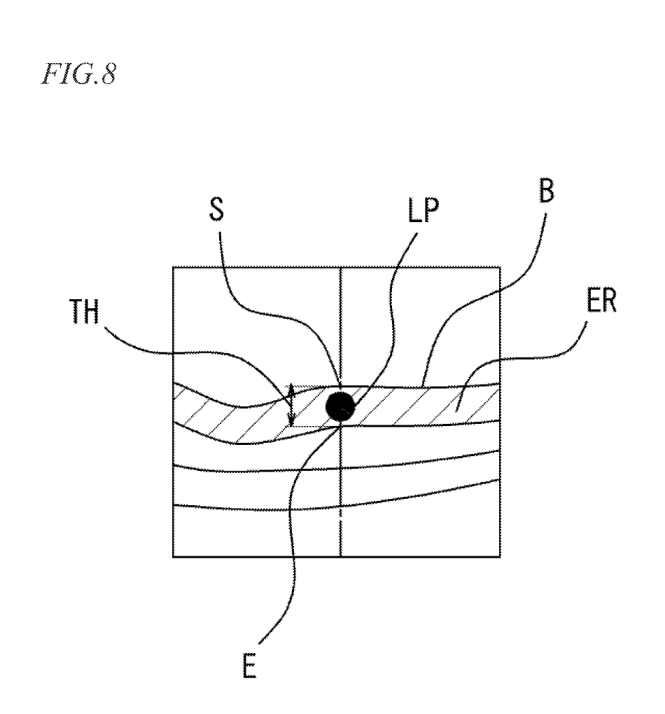

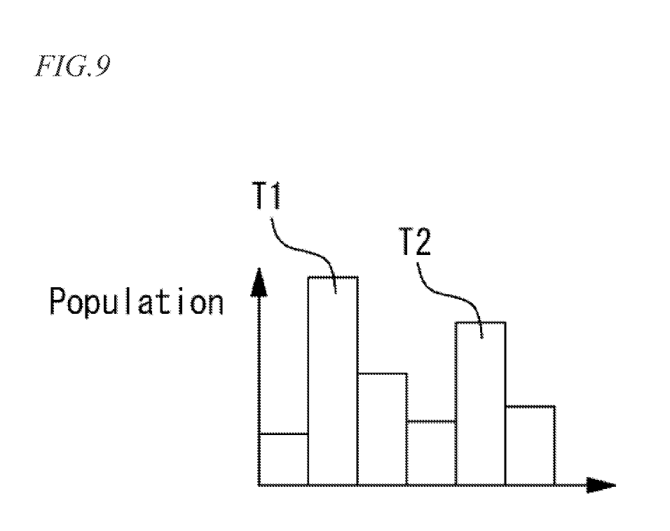

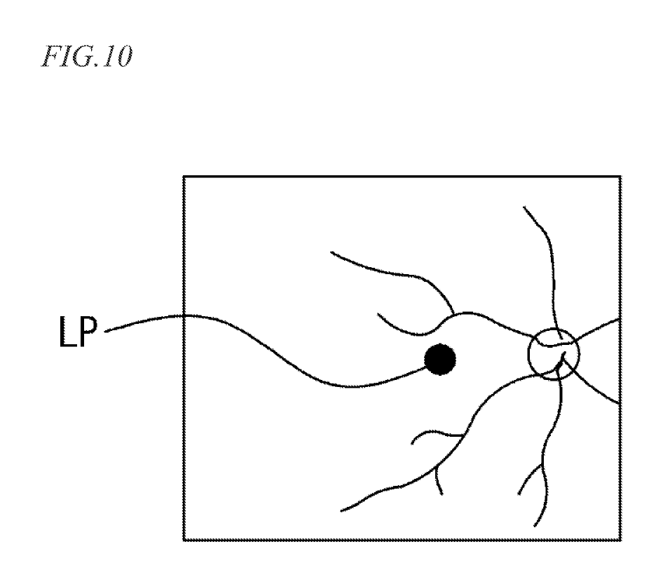

For example, as illustrated in FIG. 8, a description will be made of a case where a front image is generated in the depth region ER including a lesion portion LP such as edema in the retina. In this case, a histogram of the A scan signal corresponding to the lesion portion is as in FIG. 9. Therefore, it is assumed that a bin with the highest frequency of occurrence is a bin T1 showing luminance of the lesion portion, and a bin with the second highest frequency of occurrence is a bin T2 showing luminance of the retina around the lesion portion. In the above-described method, a median of the bin T1 with the highest frequency of occurrence is used as luminance of a front image, and the bin T2 with the second highest frequency of occurrence has no influence thereon. In other words, the luminance of the lesion portion is used in a front image, and the luminance of the retina around the lesion portion is not added thereto. Consequently, a front image of the depth region ER is an image in which the lesion portion can be clearly observed. If luminance values of the A scan signals are integrated in the depth region ER, luminance values of the retina around the lesion portion are also integrated, and thus the luminance values of the lesion portion may be obscured. In contrast, according to the method using the histogram, the luminance values of the lesion portion which takes up the depth region ER are used in respective representative luminance values of a front image, and thus the luminance values of the lesion portion are prevented from being buried. Therefore, by changing a luminance value of a front image based on such a histogram, it is possible to acquire a favorable image of a lesion portion (a defect, peeling, edema, effete matter, a neovascular lesion, or the like) LP or the like generated in the subject's eye E (refer to FIG. 10).

Even in a case where abnormal local reflection occurs in a portion whose refractive index changes, an influence of luminance with a low frequency of occurrence can be reduced by using a histogram, and thus it is possible to prevent a part of a front image from being significantly brightened due to the abnormal reflection.

<Setting of Bin Width>

In acquisition of a histogram, a bin width may be changed depending on a luminance level of the A scan signal. For example, an appropriate bin width may be automatically set according to a luminance level of the A scan signal. For example, the control unit 70 may compute a bin width for each A scan by using a difference between the maximum luminance and the minimum luminance of the A scan signal in the depth region ER. For example, in a case where a difference between the maximum luminance and the minimum luminance is great, it may be regarded that a deviation in a luminance value is considerable, and thus the quality of bins can be easily identified by increasing a bin width. On the other hand, in a case where a difference between the maximum luminance and the minimum luminance is small, it may be regarded that a deviation in the frequency of occurrence is small, and the quality of bins are easily identified, and thus grayscales of a generated image may be increased by reducing a bin width.

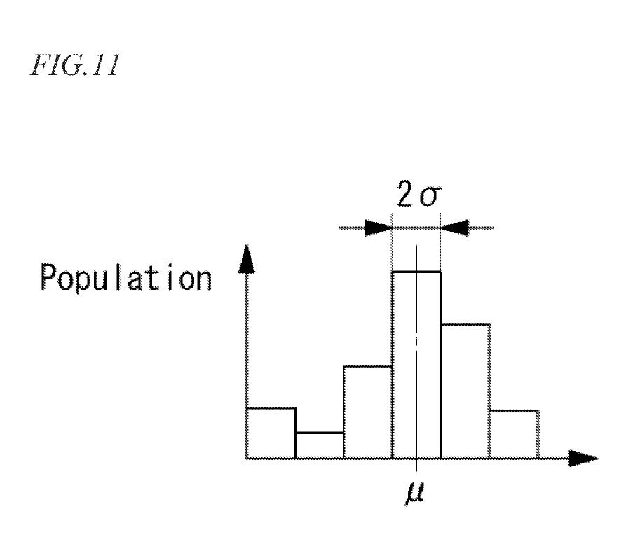

The control unit 70 may compute a bin width for each A scan by using a luminance average value and a standard deviation (or variance) of the A scan signal in the depth region ER. For example, as illustrated in FIG. 11, the control unit 70 may calculate a luminance average value μ and a standard deviation σ of the A scan signal in the depth region ER, and may set a width which is twice the standard deviation σ as a bin width with the average value as a reference.

As mentioned above, the control unit 70 can acquire a more favorable front image by setting a bin width corresponding to each A scan signal.

Regarding a representative luminance value acquired according to the above-described method, a median in a bin is used as the representative value. Therefore, grayscales of a generated front image depend on a bin width. For this reason, the control unit 70 may increase grayscales of a generated image by adjusting a representative value by using a histogram.

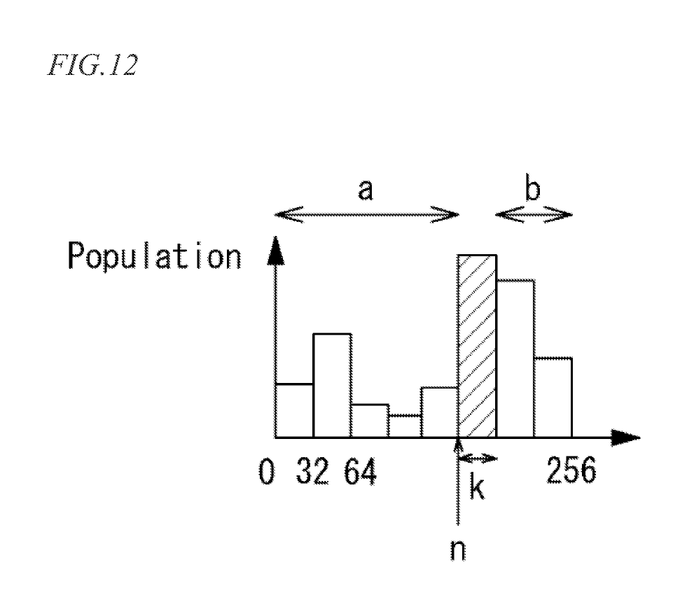

More specifically, as illustrated in FIG. 12, the control unit 70 computes an frequency of occurrence a of a luminance value smaller than the representative value and an frequency of occurrence b of a luminance value greater than the representative value. The control unit 70 changes the representative value, for example, within a range of a bin width based on a ratio of a to b. Here, when the minimum value of a bin including the representative value is denoted by n, and a bin width is denoted by k, an adjusted luminance value x is obtained by using the following equation (1).

$$\begin{matrix}[ Equation 1 ] & \\ x = n + k \times \frac{b}{( a + b )} & ( 1 )\end{matrix}$$

The representative luminance value acquired according to the method is based on the maximum frequency of a luminance value. Therefore, luminance values with the second frequency of occurrence and the subsequent frequencies of occurrence are not considered. The control unit 70 may consider luminance values with the second frequency of occurrence and the subsequent frequencies of occurrence. Consequently, a delicate change of a histogram is appropriately detected in a latent manner, and thus a more favorable front image can be acquired.

More specifically, the control unit 70 selects, for example, several high-rank bins with a high frequency of occurrence from the histogram illustrated in FIG. 4, and takes an average of representative values thereof. Thus, it is possible to acquire a more favorable front image by adding information regarding luminance values with the second highest frequency of occurrence and the subsequent frequencies of occurrence thereto. The number of high-rank bins may be arbitrarily selected. For example, bins which are equal to or higher than 50% of the frequency of occurrence of a bin with the highest frequency may be selected.

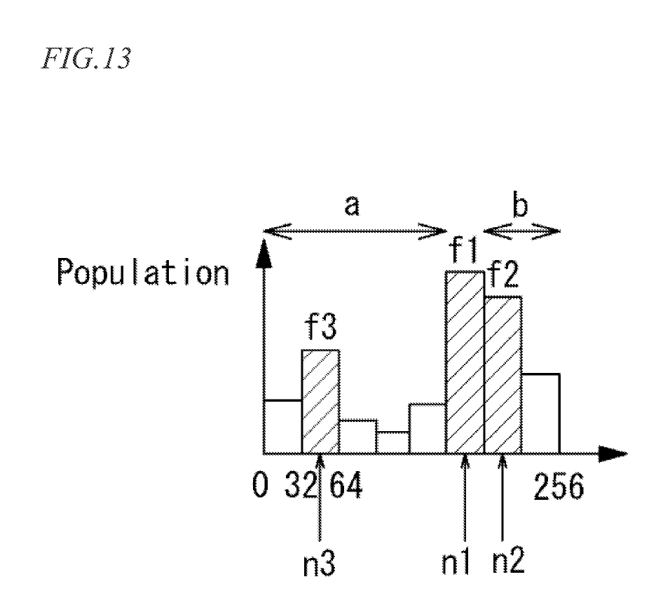

The control unit 70 can acquire a clearer front image by weighting the representative values with an frequency of occurrence and taking an average thereof. For example, as illustrated in FIG. 13, it is assumed that three bins are selected in an order of the highest frequency of occurrence in a histogram. In this case, if a representative value of a bin with the (i-th) highest frequency of occurrence is denoted by ni, and an frequency of occurrence of the bin with the (i-th) highest frequency of occurrence is denoted by fi, a weighted average value x of the three bins in an order of the highest frequency of occurrence is computed as in the following Equation (2).

$$\begin{matrix}[ Equation 2 ] & \\ x = \frac{n 1 f 1 + n 2 f 2 + n 3 f 3}{( f 1 + f 2 + f 3 )} & ( 2 )\end{matrix}$$

The optical coherence tomography apparatus 10 may generate a front image not only by acquiring three-dimensional OCT data regarding the fundus of the subject's eye E but also by acquiring the histogram from three-dimensional OCT data regarding the anterior chamber. Of course, a front image may be generated by acquiring the histogram from three-dimensional OCT data regarding parts of a biological tissue other than the eye.

What is claimed is:

1. An optical coherence tomography apparatus comprising:
a scanning unit configured to scan a test substance with measurement light;
an OCT optical system configured to detect an A scan signal caused by interference between the measurement light and reference light corresponding to the measurement light; and
a processor configured to:
acquire three-dimensional OCT data in which the A scan signals at respective scanning positions at the test substance are arranged in a two-dimensional manner;
acquire a histogram of the A scan signals at the respective scanning positions; and
generate an OCT front image by processing the acquired three-dimensional OCT data based on the acquired histogram.

2. The optical coherence tomography apparatus according to claim 1,
wherein the processor sets a luminance value of each pixel forming the OCT front image based on a luminance value which has high frequently of occurrence on the histogram.

3. The optical coherence tomography apparatus according to claim 1, wherein the test substance is a subject's eye.

4. The optical coherence tomography apparatus according to claim 1, wherein the processor acquires the histogram of the A scan signals in a specific depth region, and generates the OCT front image of the specific depth region of the test substance based on the acquired histogram.

5. The optical coherence tomography apparatus according to claim 2,
wherein the processor sets a luminance value of each pixel forming the OCT front image based on a luminance value whose frequency which occurs most in the histogram.

6. The optical coherence tomography apparatus according to claim 2,
wherein the processor sets a luminance value of each pixel forming the OCT front image based on a plurality of luminance values which have high frequency of occurrence on the histogram.

7. The optical coherence tomography apparatus according to claim 2,
wherein the processor sets a luminance value of each pixel forming an OCT front image based on a representative value of a bin of the histogram, said bin having high frequently of occurrence on the histogram.

8. The optical coherence tomography apparatus according to claim 7, wherein the processor adjusts the representative value of the bin based on the frequency of occurrence of each of bins on the histogram.

9. The optical coherence tomography apparatus according to claim 1, wherein the processor sets bin widths in such a manner that the histograms of at least the two A scan signals of which scanning positions are different from each other have difference sizes of bin widths.

10. The optical coherence tomography apparatus according to claim 1, wherein the processor sets a bin width of the histogram according to the luminance of the A scan signal.

11. The optical coherence tomography apparatus according to claim 10, wherein the processor sets the bin width of the histogram using a difference between the maximum luminance and the minimum luminance of the A scan signal.

12. The optical coherence tomography apparatus according to claim 11,
wherein the processor sets a bin width of the histogram in such a manner that the bin width of the histogram in a case where the difference is large is set larger than the bin width of the histogram in a case where the difference is small.

13. The optical coherence tomography apparatus according to claim 10, wherein the processor sets the bin width of the histogram based on a standard deviation of the A scan signal.

14. The optical coherence tomography apparatus according to claim 13,
wherein the processor sets an average luminance value of the A scan signal at a reference position of the bin, and sets a width which is twice the standard deviation as the bin width.

15. A non-transitory computer readable recording medium storing a fundus image processing program, when executed by a processor of an optical coherence tomography apparatus including a scanning unit configured to scan a test substance with measurement light and an OCT optical system configured to detect an A scan signal caused by interference between the measurement light and reference light corresponding to the measurement light, causing the optical coherence tomography apparatus to execute:
acquiring three-dimensional OCT data in which the A scan signals at respective scanning positions at the test substance are arranged in a two-dimensional manner;
acquiring a histogram of the A scan signals at the respective scanning positions; and
generating an OCT front image by processing the acquired three-dimensional OCT data based on the acquired histogram.
